(19) United States
(12) Patent Application Publication
(10) Pub. No.: US 2004/0163652 A1
(43) Pub. Date: Aug. 26, 2004

(54) Condom with restriction band

(76) Inventor: Colin Watson, Valencia, CA (US)

Correspondence Address: SHELDON & MAK, Suite 900, 225 South Lake Avenue, Pasadena, CA, 91101, US

(21) Appl. No.: 10/783,250

(22) Filed: Feb. 19, 2004

Related U.S. Application Data
(60) Provisional application No. 60/449,387, filed on Feb. 25, 2003.

(51) Int. Cl. A61F 6/04; A61F 6/02

(52) U.S. Cl. 128/844; 128/842

(57) ABSTRACT

A condom comprising a cylindrical membrane and an elastomeric restriction ring or restriction band at the open end of the condom. The restriction ring may additionally comprise a stimulation ring.

### RELATED APPLICATION

This application is a continuation-in-part of U.S. Provisional Application Serial No. 60/449,387, filed Feb. 25, 2003, the entirety of which is incorporated herein by this reference.

### FIELD OF THE INVENTION

This invention is directed generally to condoms.

### BACKGROUND OF THE INVENTION

In the last decade condom sales have grown dramatically and condom use has reached unprecedented acceptance. Due to the increased risk of AIDS and other sexually transmitted diseases, protection from such diseases has never been more important. Earlier condom products provided protection from disease but perceptibly decreased male stimulation. More recently, thinner condom materials have been developed, allowing for increased stimulation. Despite increased acceptance of such condoms, however, resistance to condom use still exists.

Other sexually-oriented devices for use by men include, inter alia, adjustable bands or rings that attach to the base of the penis to restrict blood flow from the penis back into the body. Such bands improve erectile function for people with cardiovascular problems, including those taking medication for heart disease and diabetics, and may also be used by others who are frustrated with the side effects of sexual endurance brought on by chemical enhancers of erectile function such as sildenafil citrate (sold by Pfizer under the brand name VIAGRA). Externally applied erection devices are disclosed in U.S. Pat. Nos. 4,539,980, 4,628,915, 4,378,008, 3,820,533 and Des. 258,690, the entireties of which are incorporated herein by this reference.

Several problems exist with respect to such prior art products, however. Both condoms and blood restrictive bands or rings may be difficult to attach during intercourse when needed. The time required to attach both such devices may cause a user frustration and embarrassment. The use of any additional devices to enhance intercourse, such as a soft circular ring with protruding knobs that slip over the penis to increase stimulation, would require even more time to put on and might result in further frustration.

### SUMMARY OF THE INVENTION

The condoms of the present invention address these problems by integrally combining the blood restrictive function of restriction bands or rings. In one embodiment, the condom of the invention further comprises the stimulating function of stimulation rings. The combination of these elements with a condom speeds up the attachment of these elements to a user's penis during intercourse, as compared with the attachment of each item sequentially, and thus increases user satisfaction. The addition of these enhanced features to a condom is also hoped to increase compliance with condom use among sexually active males due to the increased sexual pleasure and performance obtainable with the condoms of the present invention compared with those of the prior art, thus reducing STD's, unwanted pregnancies, and other health concerns.

### DESCRIPTION OF THE DRAWINGS

These and other features, aspects and advantages of the present invention will become better understood with regard to the following description, appended claim, and accompanying figures where:

FIG. 1 is a partial sectional view of the open end of one embodiment of the condom of the present invention.

FIG. 2 is a side view of an alternative embodiment of the condom of the present invention.

FIG. 3 is a sectional view of the open end of the condom of FIG. 2 along line B-B.

FIG. 4 is a plan view of the open end of the condom of FIG. 1.

FIG. 5 is a sectional view of FIG. 4 along line A-A.

### DETAILED DESCRIPTION

The following discussion describes in detail one embodiment of the invention and several variations of that embodiment. This discussion should not be construed, however, as limiting the invention to those particular embodiments. Practitioners skilled in the art will recognize numerous other embodiments as well.

The condoms of the present invention comprise an elastic cylindrical membrane 2 of a generally conventional size and shape having an open end 3 and a closed end 4. The cylindrical membrane 2 may be either of the roll-up type or of accordion type construction and is typically made of an elastic material such as latex, polyurethane or other suitable material. The condom is disposable and is removed and discarded in the usual fashion after use.

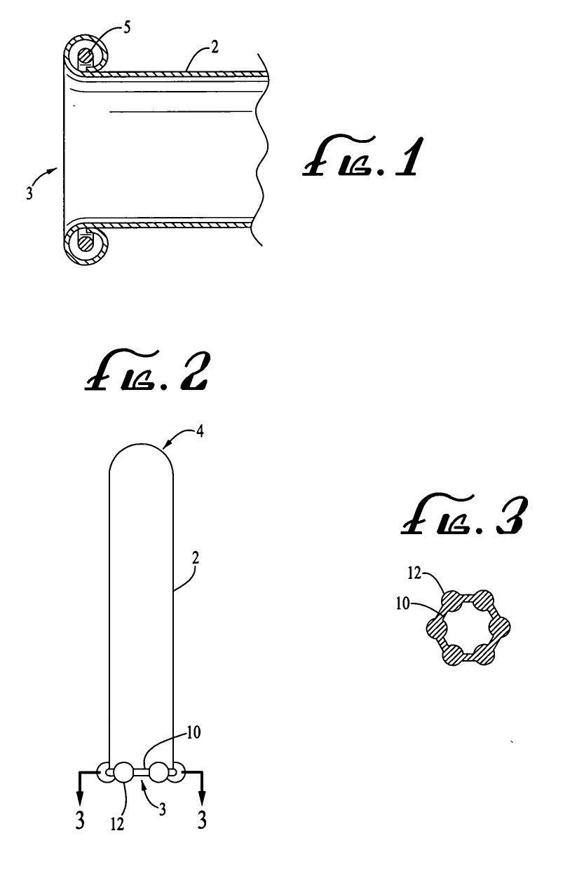

In the invention, the condom is provided with a closed-loop, elastomeric restriction ring 5 which serves to retain the condom on the penis during coitus and to restrict the flow of blood from the erect penis back into the body, thereby facilitating the maintenance of an erection by the user (see FIG. 1). The restriction ring 5 is securely attached to or integrally molded with the open end 3 of the condom. The restriction ring is flexible and elastic such that it can be stretched wide enough to allow a penis to pass through it, but must be of a width such that upon relaxation it grips the penis when positioned at the base and provides sufficient pressure to prevent blood from freely flowing from the penis back into the body, thereby allowing for the maintenance of an erection for longer periods of time.

The restriction ring 5 is preferably molded from an elastomeric material such as natural rubber, silicone, or other synthetic rubber having a durometer number in the range of 40 to 60, with a durometer number of about 55 being preferred. Other useful materials which may be used include plastics such as polyurethane, polyvinyl chloride or polyethylene. Rings of different diameters may be provided to accommodate individual differences between users.

The restriction ring 5 in this embodiment may be disguised or camouflaged so as to appear to be part of a standard condom lacking additional features beyond an elastic membrane. This embodiment eliminates the embarrassment of explaining the purpose of a restriction ring to a partner with whom a user of the present invention has not previously been intimate. For example, as shown in FIG. 1, the restriction ring 5 might be attached to the open end 3 of the condom, whereby a portion of the cylindrical membrane 2 at the open end 3 is rolled around the restriction ring 5. The restriction ring is then sealed within the cylindrical membrane 2 by means of a suitable adhesive, heat, or other means known to the art. Since conventional roll-up type condoms generally terminate at the open end in a thin ring of the membrane material, this configuration serves to camouflage the presence of the restriction ring 5. The restriction ring 5 may also be otherwise attached to an exterior surface of the cylindrical membrane 2 adjacent the open end 3 or more preferably to an interior surface, in order to camouflage its presence, or may be integrally molded with the cylindrical membrane 2.

In an alternative embodiment (not shown), the restriction ring is composed of a wider band of elastic material (i.e., a restriction band) which can be stretched wide enough to allow a penis to pass through it but upon relaxation grips the base of the penis and provides sufficient pressure to prevent blood from freely flowing from the penis back into the body. Such a restriction band may be camouflaged by being incorporated into and integrally molded with the cylindrical membrane 2 or may be otherwise attached to the interior or exterior surface at the open end 3 of the condom. If disposed on the exterior surface of the condom, the band may be colored or carry a decorative design to disguise its purpose.

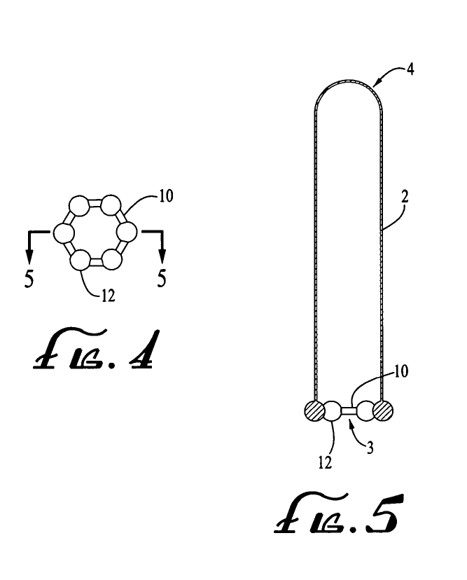

In another embodiment of the present invention (shown in FIGS. 2-5), the condom further comprises one or more protrusions 12, preferably dome- or knob-like or of spherical contour, located on and attached to a stimulation ring 10 which is securely attached to or integrally molded with the open end 3 of the condom. When a plurality of protrusions 12 are present, they are preferably arranged in a regular circumferential array in the region of the open end 3 of the condom and disposed about the outer periphery of the stimulation ring 10, and are preferably located immediately adjacent the open end 3 of the condom.

The protrusions 12 in one embodiment are spherical, and preferably have a diameter of approximately 0.15 inches (3.81 millimeters). However, the diameter of the protrusions 12 could be in the range of 0.07 to 0.3 inches (1.78 to 7.62 millimeters) or more, and the same condom can be provided with protrusions of different sizes or shapes. The protrusions 12 may also extend further radially from the stimulation ring 10. The stimulation ring 10 and protrusions 12 are preferably made of an elastic, impermeable material such as natural rubber, synthetic rubber such as silicone rubber, or plastics such as polyurethane, polyvinyl chloride or polyethylene.

In a preferred embodiment, the stimulation ring 10 may also function as a restriction ring 5 as described above. The presence of the protrusions 12 on the restriction ring 5 in this embodiment serves the dual purposes of providing increased stimulation and also obscuring the presence or purpose of the restriction ring. In this embodiment the protrusions may be further sized or shaped such that they may be gripped by the fingers of a user, thereby facilitating the widening of the restriction ring for placement over the penis of the user and also facilitating final placement of the restriction ring at the base of the penis.

In an exemplary embodiment of the present invention, the cylindrical membrane 2 of the condom will have a thickness in the range of 0.11 millimeters+/−0.04 millimeters so as to avoid dulling sensitivity. The width of the cylindrical membrane is approximately between 44 and 56 millimeters (though the open end 3, if attached to a restriction ring, may be narrower). Multiple lengths may be provided, generally in the range of 180+/−20 millimeters, though shorter or longer lengths may also be used. In one embodiment a length of 126 mm (approximately 5 inches) is used. The condom may also optionally be provided with a lubricant and/or spermicide on its outer surface.

To make a condom, a glass form or other former of conventional shape is dipped one or more times into liquid polymer baths, e.g. two baths of latex or liquid polyurethane, and then withdrawn. This leaves the form coated with the polymer liquid which, when cured, will become the cylindrical membrane 2. Between dips a restriction ring 5, restriction band (not shown), or stimulation ring 10 may be placed on what will become the open end 3 of the cylindrical membrane 2 of the condom in order to incorporate (i.e. integrally mold) it with the cylindrical membrane 2. Protrusions 12 may be integrally molded with the restriction ring 5 or stimulation ring 10 or, alternatively, may be formed from a relatively viscous material, e.g. thickened latex, and applied by a dispensing gun. If the form carrying a ring and/or protrusions 12 is subsequently dipped into a further liquid polymer bath, then the ring and any protrusions are overmolded and are held between layers of the condom material.

The polymer material on the form is then cured to form the final condom. If a restriction ring 5, restriction band (not shown), or stimulation ring 10 has not been already integrated into the condom, then such ring or band may be attached after curing, using an appropriate adhesive or a heating step, if appropriate. After curing of the condom material, the open end 3 of the cylindrical membrane 2 may be rolled downward along the form to form a roll-up type condom.

Although the present invention has been discussed in considerable detail with reference to certain preferred embodiments, other embodiments are possible. Therefore, the scope of the appended claims should not be limited to the description of preferred embodiments contained in this disclosure.

What is claimed is:

1. A condom comprising:
(a) an elastic, cylindrical membrane having a closed end and an open end; and
(b) an elastomeric restriction ring attached to said open end of said cylindrical membrane, wherein said ring is adapted to fit over an erect human penis when stretched, and wherein said ring is further adapted to fit around the base of an erect human penis and place pressure thereon when relaxed, thereby constricting the flow of blood from said penis.

2. The condom of claim 1, wherein said restriction ring is integrally molded with said open end of said cylindrical membrane.

3. The condom of claim 0.1, wherein said restriction ring is attached to a surface of said cylindrical membrane.

4. The condom of claim 1, wherein said restriction ring is made from a material selected from the group consisting of natural rubber, silicone, polyurethane, polyvinyl chloride and polyethylene.

5. The condom of claim 1, wherein said restriction ring comprises a band of elastic material.

6. The condom of claim 5, wherein said band is integrally molded with said open end of said cylindrical membrane.

7. The condom of claim 5, wherein said band is attached to an interior surface of said open end of said cylindrical membrane.

8. The condom of claim 1, wherein said restriction ring further comprises protrusions disposed about an outer periphery of said restriction ring.

9. The condom of claim 8, wherein said protrusions are arranged in a regular circumferential array.

10. The condom of claim 8, wherein said protrusions are knob-like, dome-like or of spherical contour.

11. The condom of claim 8, wherein said protrusions have a diameter in the range of 0.07 to 0.3 inches.

12. The condom of claim 11, wherein said protrusions have a diameter of approximately 0.15 inches.

13. The condom of claim 8, wherein said protrusions may be gripped by fingers.

14. The condom of claim 1, wherein said condom is provided with a lubricant or spermicide on its outer surface.

15. The condom of claim 1, wherein said cylindrical membrane is formed from a material selected from the group consisting of latex and polyurethane.

16. A condom comprising:
(a) an elastic, cylindrical membrane having a closed end and an open end;
(b) an elastomeric restriction ring attached to said open end of said cylindrical membrane, wherein said ring is adapted to fit over an erect human penis when stretched, and wherein said ring is further adapted to fit around the base of an erect human penis and place pressure thereon when relaxed, thereby constricting the flow of blood from said penis; and
(c) protrusions disposed about an outer periphery of said restriction ring.

17. The condom of claim 16, wherein said protrusions are arranged in a regular circumferential array.

18. The condom of claim 16, wherein said protrusions are knob-like, dome-like or of spherical contour.

19. The condom of claim 16, wherein said protrusions have a diameter in the range of 0.07 to 0.3 inches.

20. The condom of claim 16, wherein said condom is provided with a lubricant or spermicide on its outer surface.
